(19) United States
(12) Patent Application Publication
(10) Pub. No.: US 2002/0183726 A1
(43) Pub. Date: Dec. 5, 2002

(54) Hand piece for cosmetic or medical laser treatment

(76) Inventors: Jens Elbrecht, Jena (DE); Michael Schmidt, Feldkirch-Gisingen (AT); Eckhard Schroeder, Eckental (DE); Frank Weidner, Loerschuetz (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/148,783

(22) PCT Filed: Dec. 7, 2000
(86) PCT No.: PCT/EP00/12321

(30) Foreign Application Priority Data
Dec. 8, 1999 (DE) 199 59 698.0

(51) Int. Cl. A61B 18/20

(52) U.S. Cl. 606/6; 607/89; 606/9; 606/13

(57) ABSTRACT

The invention relates to a hand piece which is used to manually direct a laser beam onto biological tissue for the purpose of cosmetic or medical treatment A start/stop signal transmitter (11) for a laser beam source (7) integrated into the inventive hand piece and at least one control device (12) for preselecting and/or changing the physical parameters of the laser beam (3) is also integrated therein. The inventive device allows an operator to activate and regulate the laser independently of a foot pedal device. When the parameters have to be adjusted during treatment, the operator no longer needs to avert his or her gaze from the treatment site and look at a control module placed at a distance from the treatment site.

### FIELD OF THE INVENTION

The present invention relates to a hand piece which is used to manually direct a laser beam onto biological tissue for the purpose of cosmetic or medical treatment.

### RELATED ART

In cosmetics and in medicine, energy is frequently coupled into selected regions of the biological tissue with the aid of laser radiation to thereby selectively produce changes in the tissue. Thus, for example, in dermatology, laser radiation is used to remove tattoos, for the treatment of salmon patches, for hair removal, etc. But the use of laser radiation has also proven to be efficient in eye medicine, in particular, in intraocular cataract surgery. In this context, the goal of the intraocular intervention is to the remove the eye lens, which is clouded, for example, by the senile cataract, from the lens capsule. Using the laser radiation, the clouded eye lens is destroyed step by step and removed through an incision in the tissue of the anterior chamber.

Especially in the latter case, it is required to introduce the energy in a well-directed and quantitatively regulated manner so as to produce a defined division or separation of tissue. The separated lens material must be removed from the treatment site, which is generally accomplished through the cannula of a suction/rinsing system. To avoid an impairment of neighboring tissue structures, it is necessary for the clouded eye lens to be accurately divided into pieces and removed which, making allowance for individually different tissue properties, requires a very precise preselection of the laser parameters upon estimation of the treatment to be carried out and an accurate control of the laser parameters during the application of the laser radiation, in particular, with regard to the energy to be introduced into the tissue. To this end, the operator must have at his/her disposal instruments which allow simple control and precise handling.

In the case of phacoemulsification using ultrasound, a device technology has been developed which is generally constituted by a hand piece designed for introducing the energy into the patient's eye, a control module, and a foot pedal unit. An equipment of that kind is described in U.S. Pat. No. 5,580,347. It follows from this specification that the three modules hand piece, control module and foot pedal unit are spatially separated from each other. In this context, the control module is equipped with at least one operating control element for setting at least one parameter, inter alia, specifically for setting the energy.

The foot pedal unit contains a foot pedal which allows control of the actions intended by the operator, namely in accordance with the parameters which are preselected and, possibly, also changed during the treatment via the control module. The foot pedal can be positioned as a separate module in such a manner that during treatment, the operator, by placing a foot onto a rocker and changing the inclination thereof by foot movement, the energy which is introduced into the eye can be varied in its intensity up to a maximum which is preselected with the operating control element on the control module.

Consequently, the operator has to control three device units: the control module for preselecting and, possibly, also for adjusting parameter ranges during the operation, the foot pedal for quantitatively regulating the parameters within the preset ranges, and the hand piece which is to be precisely aligned by hand for introducing the energy in a well-directed manner.

In addition, there is also the fact that the separated tissue particles have to be removed from the surgery site through a suction cannula, as already indicated above. For this purpose, additional devices are required, namely a suction device having a vacuum source which is connected via a hose line to the suction cannula, which is frequently attached to or integrated in the hand piece. Here too, there are operating control elements which enable the operator to switch on and off the suction device and, possibly, also to vary the suction vacuum during the surgery to adapt the speed of withdrawal of the arising quantity of particles.

Therefore, the operator has to control a plurality of devices placed at different locations and, in addition, to monitor them during the surgery, which finally hinders him/her from intently handling the hand piece and from precisely directing the energy into the preselected target region.

German Patent Application DE 198 11 627 A1 describes a device for treating objects with laser radiation which is designed, in particular, for skin treatment. Here too, the laser beam is directed onto the skin region to be treated with the aid of a hand piece, the hand piece having an operating control device which allows the setting of laser radiation parameters. However, the setting capacity relates to the sequence of laser pulses having different energies and to the degree of overlapping of the spots at the location of incidence; no provision being made for a device for activating and stopping the laser radiation.

Furthermore, the Asah Medico A/S Company, Valseholmen 11-13, DK-2650 Hvidovre, Denmark, offers a laser hand piece under the name “MedArt®” which is designed for laser scanning and equipped with an operating control device for influencing the scanning patterns, the scanning width and the scanning time. Here too, it is not possible to activate or stop the laser radiation from the hand piece.

### DESCRIPTION OF THE INVENTION

Starting from this related art, the object of the present invention is to further develop a hand piece of the type specified at the outset in a manner allowing physical parameters of the laser radiation to be preselected and changed while concentrating on the treatment site in an undiminished manner.

According to the present invention, a start/stop signal transmitter for the laser radiation source as well as at least one control device for preselecting and/or changing physical parameters of the laser radiation are integrated in a hand piece of the type described above. In this manner, it is achieved that the operator is not forced to avert his/her gaze from the treatment site and direct it toward a control module placed at a distance from the treatment site when parameters or parameter limits have to be adjusted during treatment.

Using the hand piece according to the present invention, there is also no need to share attention between the handling of the hand piece and the actuation of the foot pedal but it is possible to carry out all required steps directly at the hand piece, including the alignment of the hand piece with the target region, the switching on and off of the radiation source and the regulation of the physical parameters of the radiation, and to concentrate attention exclusively on the treatment site.

In a preferred embodiment, the control device integrated in the hand piece is designed for changing the pulse frequency of the laser radiation. In the cataract surgery described, a pulsed laser beam is directed towards the treatment site so that the laser power can be varied by changing the pulse frequency using a control device designed in such a manner. Alternatively, given a suitably modified design of the control device, it is also possible to increase or reduce the energy of the laser radiation while the pulse frequency remains constant. The laser power can be regulated in this manner as well.

It is advantageous if the start/stop signal transmitter and the control device are assigned a common operating control element which is to be moved over the length of a first actuator travel for triggering the start/stop signal and over the length of a second actuator travel for changing physical parameters of the laser radiation. In this context, the operating control element is advantageously positioned on the hand piece in such a manner that it can be moved over the first and also the second actuator travels by the thumb or index finger of the hand that holds the hand piece without having to change the alignment of the hand piece relative to the treatment site in the process. Thus, it is not required for the operator to avert his/her gaze from the treatment site, that is, he/she does not need to monitor the manipulation of the start/stop signal transmitter or of the control device. Given an ergonomic design of the actuator travel lengths, of the adjustment force, of the shape of the operating control element, etc., precise starting of the laser radiation source and regulation of the pulse frequency of the laser radiation are possible through the sense of touch alone within the shortest phases of habituation.

In this context, the actuator travels can have the same direction so that the operating control element is first moved over the first actuator travel length, the start signal being triggered in the process, and provision being for the operating control element to be subsequently moved further in the same direction. In special embodiments, provision can be made for the displacement over the second actuator travel length to be enabled only when the operating control element has previously been moved over the entire first actuator travel length. For this, it is possible to use a simple mechanical lock as it is known in the related art from the most different kinds of devices.

It is also within the scope of the present invention that the two actuator travels are oriented orthogonally to each other, the operating control element being initially moved in one direction over the first actuator travel length, triggering the start signal in the process, and the displacement over the second actuator travel length being possible, namely in an orthogonal direction, only after the displacement over the entire first actuator travel length.

In an embodiment which has already proven efficient in practice, the first actuator travel has a length of 2 to 10 mm and the second actuator travel has a length of 0.1 to 1 mm. In other words: The operating control element is initially displaced over a length of at least 2 to at most 10 mm, triggering the start pulse for the laser radiation source in the process. At the end of the first actuator travel or after triggering the start pulse and starting the laser radiation source, the path for the displacement of the operating control element over the second actuator travel is now cleared, it being possible to move the operating control element over a length of at least 0.1 to at most 1 mm and, in doing so, to change the pulse frequency.

It is advantageous for the pulse frequency to be changed in proportion to the actuator travel length. That is, the larger the displacement of the operating control element over the second actuator travel, the higher is the set pulse frequency and, consequently, also the laser power. Thus, the actuator travel length and the pulse frequency can be matched to each other in such a manner the displacement of the operating control element over the range from 0.1 to 1 mm will result in a change in the pulse frequency from 1 to 200 Hz and, in this manner, in a change in the laser power approximately within the range from 10 mW to 2 W.

Moreover, an advantageous technical embodiment consists in that the second actuator travel, i.e., the displacement of the operating control element over the second actuator travel, is converted into adequate pressure values of a medium. This is achieved, for example, by moving the operating control element in the direction toward a pressure vessel, whereby the action of force on a membrane which, in turn, acts upon the medium enclosed within the pressure vessel is increased as the actuator travel length increases, as a result of which the pressure inside the vessel is increased. Using a second converter, the pressure values can be converted into adequate analog electrical control signals and used to change the pulse frequency via a laser driving circuit.

Furthermore, it is conceivable to integrate into the hand piece a second start/stop signal transmitter for a suction device as well as a further control device for preselecting and/or changing the suction performance of the suction device. A hand piece of that kind allows the operator to influence both the parameters of the laser radiation and the quantity of separated tissue particles to be drawn off per unit of time. Thus, it is possible for the operator to match the laser power and the suction performance to each other without the need to reduce his/her concentration on the treatment site.

In further preferred embodiments of the present invention, the start/stop signal transmitter(s) and control device(s) are indeed integrated in the hand piece but designed as a module which is connected to the remaining parts of the hand piece only frictionally and/or positively and which is detachable by hand force, possibly involving two-handed manipulation. This embodiment advantageously serves to allow the module containing the start/stop signal transmitters and the control devices to be separated in an uncomplicated manner, for example, for the purpose of sterilization. With regard to this, this module of the hand piece containing the start/stop signal transmitters and control devices should be made from materials which allow sterilization to be carried out without the risk of damage.

The present invention is not limited to preselecting and changing the pulse frequency of laser radiation which is to be introduced into the tissue in a pulsed manner. Moreover, it is included in the present invention and conceivable to allow further physical parameters such as pulse length, pulse shape, pulse width modulation, laser wavelength, etc. to be influenced using the control device. In this context, work under sterile conditions can always be accommodated in that the start/stop signal transmitter and control device are detachable from the hand piece and able to be sterilized in the described manner.

It is, of course, also possible to conceive of devices which are indeed provided with a hand piece having an integrated control device for one or more laser parameters but which nevertheless still include a foot pedal which is provided to the operator for optional use or which makes it possible to preselect or change further parameters which cannot be influenced using the control device on the hand piece.

In further embodiments of the present invention, the control device integrated in the hand piece can also be designed in the form of a microphone so that the operator can start the laser radiation source and/or preselect or change physical parameters, for example, by command sounds. In doing so, an analog electrical output signal is generated via the microphone, for example, as a function of the volume or sound frequency, it being possible to use the output signal for influencing selected parameters via the laser driving circuit in the manner described above. Along these lines, it is also possible to conceive of further control devices which are able to be controlled by the mouth of the operator (tongue pressure, puffing, biting pressure). In this context, all devices are interesting and usable which allow the laser radiation parameters to be influenced independently of the foot switch.

In connection with the converter of pressure values into adequate analog electrical or electronic control signals described further above, provision can be made to convert, for example, three pressure values which each correspond to a threshold value into three adequate electrical signals. This results in the possibility for three successive pressure levels or electrical control signals which are adequate to these pressure levels to be obtained and further processed as a function of increasing pressure values. These pressure levels or control signals can be assigned specific frequencies, power, pulse lengths, pulse shapes, pulse widths, laser wavelengths, etc., which can be invoked in this manner.

### BRIEF DESCRIPTION OF THE DRAWING

In the following, the present invention will be explained in greater detail in the light of an exemplary embodiment.

In the appertaining drawings,

FIG. 1 shows an overview of the hand piece according to the present invention, including the coupling to a laser radiation source;

FIG. 2 is cutaway portion A from FIG. 1, including a detail which is a schematic representation of an operating control element in the unoperated position;

FIG. 3 shows the same detail A from FIG. 1 with the operating control element having covered the first actuator travel;

FIG. 4 shows the same detail A from FIG. 1 with the operating control element having covered the second actuator travel;

FIG. 5 shows a design variant of the operating control element.

### DETAILED DESCRIPTION OF THE DRAWING

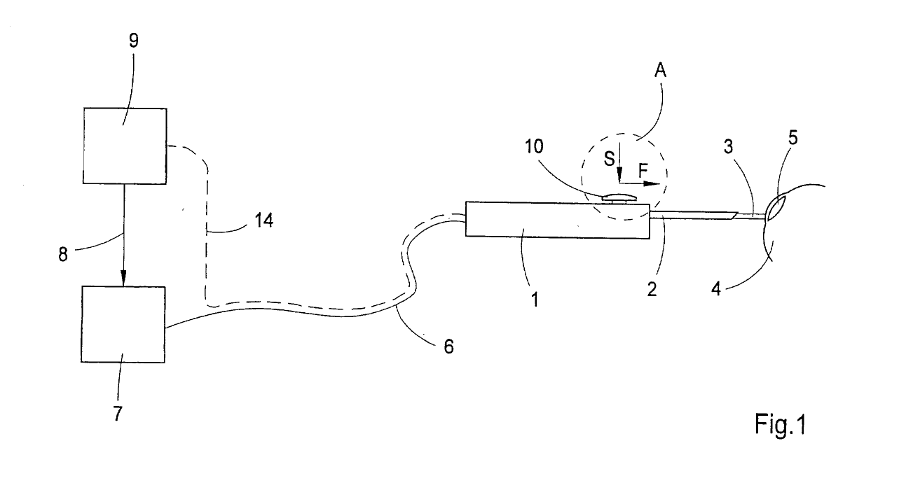

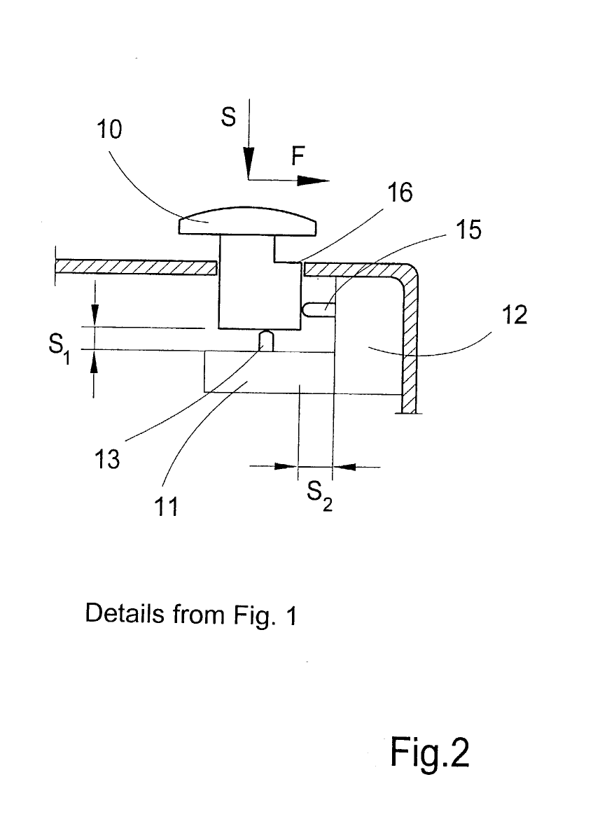

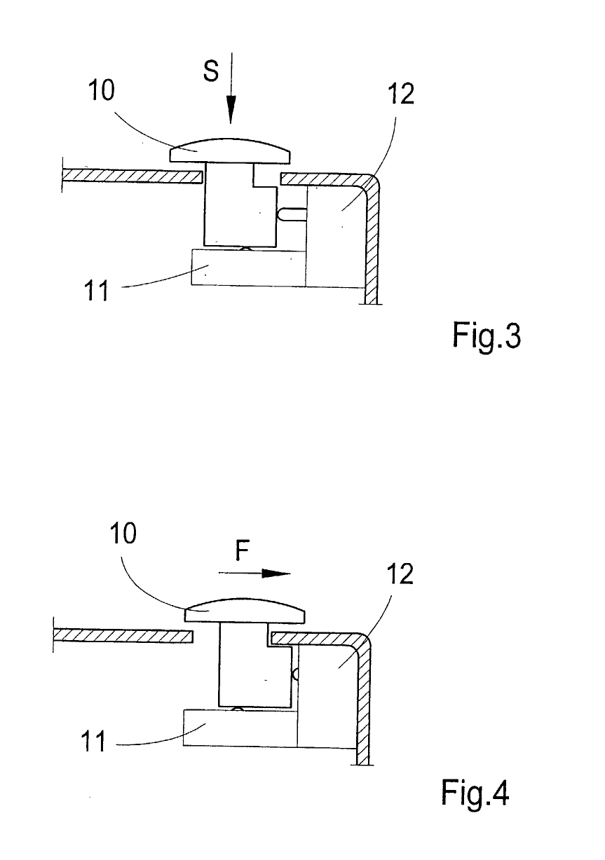

In the following, the present invention will be explained by the example of a hand piece for intraocular cataract surgeries according to the method of phacoemulsification which is known per se. In this context, FIG. 1 shows a hand piece 1 which is equipped with a phacotip 2 from which emerges a laser radiation 3 which is directed toward the lens of an eye 4. As already explained, the goal of the surgical intervention is to destroy eye lens 5 step by step and to remove the tissue particles which are in each instance separated through an incision in the anterior chamber.

As a rule, the removal is accomplished by suction using a suction cannula which is directed toward the surgery site and connected to a suction device. The suction cannula and the suction device are not graphically depicted but sufficiently known from the related art.

Hand piece 1 is connected to a laser radiation source 7 via an optical fiber 6. Provided as laser radiation source 7 is, for example, an Er:YAG laser.

Laser radiation source 7, in turn, is connected to a laser driving circuit 9 via a signal path 8. The setting of parameters for laser radiation 3 can be carried out via laser driving circuit 9. This includes above all the influencing of the laser power and thus, of the energy input into the tissue. In principle, two ways are conceivable for this, in which, provided that laser radiation 3 is pulsed, either the pulse frequency is changed, thereby varying the laser power, or the energy of laser radiation 3 is changed while the pulse frequency is constant.

In the related art which has already been explained, it has heretofore been usual to only equip laser driving circuit 9 with at least one operating control element for setting at least one parameter of the laser radiation. Since laser driving circuit 9 and hand piece 1 are spatially separated from each other, the operator would need to turn toward the laser driving circuit 9 for presetting or changing the laser beam parameters, reducing his/her concentration on the site of the surgical intervention. However, this makes the treatment more difficult for the operator, increasing the risk to the patient.

To eliminate this shortcoming, provision is made according to the present invention to integrate into the hand piece an operating control element 10 which is connected both to a start/stop signal transmitter 11 (compare FIG. 2) and to a control device 12 for changing the pulse frequency of laser radiation 3.

As indicated in FIG. 2, the operating control element is supported in straight-line guides which initially allow a displacement in direction S and thereupon a displacement in direction F. Upon displacement in direction S, a start signal for laser driving circuit 7 is triggered via laser driving circuit 9. The displacement in direction F changes, for example, the pulse frequency, it being preferred to carry out a change in the range from 1 Hz to 200 Hz.

In other words: when pressure is exerted upon the upper side of operating control element 10 in direction S, the operating control element is displaced by an actuator travel s₁, whereby an electrical contact is closed via a pushbutton plunger 13 inside start signal transmitter 11, thereby outputting a piece of information via control line 14 to laser driving circuit 9 which thereupon causes laser radiation source 7 to be switched on. When displacing operating control element 10 in direction F, a pushbutton plunger 15 is displaced over an actuator travel s₂, thereby also transmitting actuating signals via control line 14 to laser driving circuit 9. In this connection, control device 12 is designed in such a manner that the displacement of operating control element 10 over actuator travel s₂, which advantageously has a length in the range from 0.2 to 1 mm, will proportionally change the pulse frequency from 1 Hz to 200 Hz, that is, the actuator travel length of 0.1 mm is assigned a pulse frequency of 1 Hz, the actuator travel length of 1 mm is assigned a pulse frequency of 200 Hz. Between these ranges, the actuator travel and the pulse frequency change in proportion to each other.

With regard to this, control device 12 can be designed in such a manner that the change in position of pushbutton plunger 15 produced via operating control element 10 causes a force to act upon a membrane which is integrated in the outside wall of a pressure vessel filled with a medium. The action of force on the membrane increases with increasing change in position of pushbutton plunger 15 over actuator travel s₂, as a result of which the internal pressure in the pressure vessel increases as well. Namely also in proportion to actuator travel length s₂ which is covered respectively. This pressure increase can be converted into analog electrical signals via a converter which is integrated in control device 12 but not graphically depicted separately. That is, the higher the pressure inside the pressure vessel, the higher or lower (depending on the circuit design) are the current or voltage values which are transmitted to laser driving circuit 9 via control line 14. The pulse frequency of laser radiation 3 output by laser radiation source 7 is increased or reduced by laser driving circuit 9 as a function of these current or voltage values.

In this manner, the operator has the possibility of both starting laser radiation source 7 and quantitatively regulating the laser power in a precise manner without the need to avert his/her attention from the operation site or from the patient.

In this context, moreover, a particularly preferred design variant consists in that displacement directions S and F are locked with respect to each other and, because of this, are dependent on each other. In the concrete case, this is accomplished by a stop edge 16 which makes it impossible for operating control element 10 to be moved in direction F before actuator travel s₁ has been covered in direction S. Using this arrangement, it is at the same time possible to accomplish safeguards which require a stepwise switching on of laser radiation source 7. Thus, for example, it is conceivable for further pushbutton plungers to be arranged next to pushbutton plunger 13, possibly also next to pushbutton plunger 15, the further pushbutton plungers also being actuated by moving operating control element 10 in directions S and F, thereby operating switching contacts which are able to be used for triggering corresponding actuating signals.

At the end of the surgery, the operator moves operating control element 10 back in an opposite direction to drawn-in displacement direction F in the course of which the pulse frequency is reduced, whereupon operating control element 10 can be released. In the process, the resetting takes place in an opposite direction to drawn-in displacement direction s, and the stop signal is triggered. In this context, technical design variants are, of course, possible in many ways which, for example, cause operating control element 10 to be reset over actuator travels s₁, and s₂ through spring force.

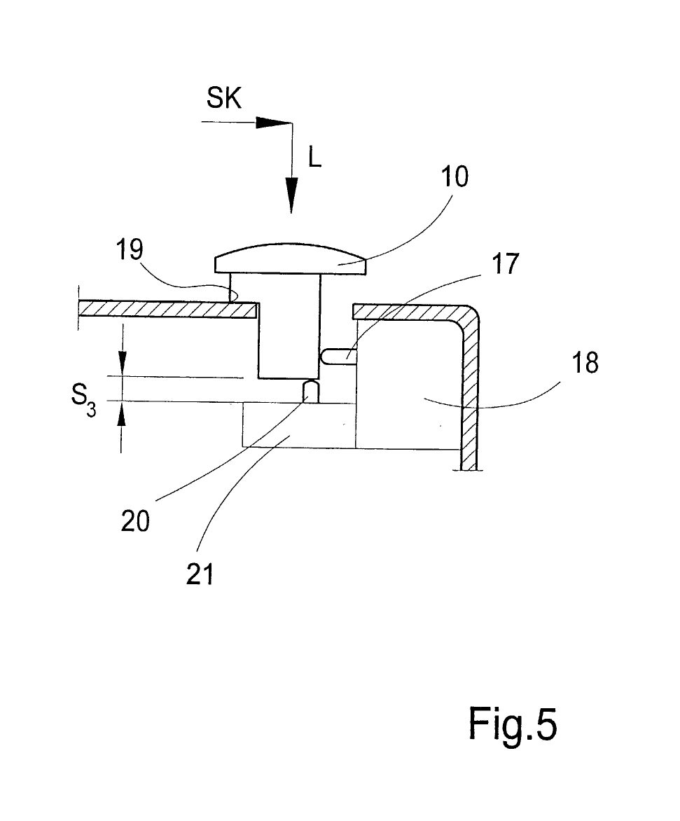

The present invention is, of course, not exclusively limited to the features described in the above exemplary embodiment. Thus, moreover, an embodiment is conceivable as is shown in FIG. 5. There, operating control element 10 is designed in such a manner that initially, a displacement is possible only in direction SK and that a displacement in direction L is possible only after that.

Through the displacement in direction SK, initially pushbutton plunger 17 of a switch 18 is actuated which closes or opens the shutter circuit of the laser radiation source. Stop edge 19 clears the path for the displacement in direction L only after the displacement in direction SK, the shutter circuit, however, being already closed and pushbutton plunger 20 of a control device 21 being actuated now. Pushbutton plunger 20 is actuated in direction L over actuator travel s₃, in the course of which initially the laser radiation is triggered via a switching contact (which is not graphically depicted) and, thereupon, the pulse frequency of the laser radiation is continuously or progressively changed in the course of the remaining rest of actuator travel s₃.

What is claimed is:

1. A hand piece for cosmetic or medical treatment from which a laser radiation (3) is manually directed onto a treatment site,
characterized by a start/stop signal transmitter (11), which is integrated in the hand piece (1), for the laser beam source (7) as well as at least one control device (12), which is/are also integrated in the hand piece (1), for preselecting and/or changing physical parameters of the laser radiation (3).

2. The hand piece as recited in claim 1,
wherein the start/stop signal transmitter (11) and the control device (12) are assigned a common operating control element (10), it being possible to move the operating control element (10) over the length of a first actuator travel (s1) to trigger the start/stop signal and over the length of a second actuator travel (s2) to change physical parameters of the laser radiation.

3. The hand piece as recited in claim 2,
wherein the two actuator travels (s1, s2) are dependent on each other, the ability of displacement over the second actuator travel (s2) requiring the completion of the displacement over the first actuator travel (s1) and, thereby, the triggering of the start signal and, conversely, the resetting of the operating control element (10) over the first actuator travel (s1) and, thereby, the triggering of the stop signal requiring the completion of the resetting of the operating control element (10) over the second actuator travel.

4. The hand piece as recited in claim 2 or 3,
wherein the operating control element (10) is displaceably supported in straight-line guide elements, the first and second actuator travels (s1, s2) being oriented orthogonally to each other.

5. The hand piece as recited in one of the preceding claims,
wherein the first actuator travel (s1) has a length of 2 to 10 mm and the second actuator travel (s2) has a length of 0.1 to 1 mm.

6. The hand piece as recited in one of the preceding claims,
wherein a pulsed laser radiation (3) is directed onto the tissue, and the control device (12) is designed for changing the pulse frequency of the laser radiation (3) within a preselected pulse frequency range.

7. The hand piece as recited in one of the preceding claims,
wherein provision is made for a second control device for preselecting the pulse frequency range for a specific treatment.

8. The hand piece as recited in one of the preceding claims,
wherein the displacement of the operating control element (10) over the first actuator travel (s1) triggers the start signal, the displacement of the operating control element (10) over the second actuator travel (s2) increases the pulse frequency within a pulse frequency range from 1 to 200 Hz, the resetting of the operating control element (10) in the opposite direction over the second actuator travel (s2) reduces the pulse frequency within the pulse frequency range from 1 to 200 Hz, and the resetting of the operating control element (10) in the opposite direction over the first actuator travel (s1) triggers the stop signal.

9. The hand piece as recited in claim 8,
wherein the change in the pulse frequency within the pulse frequency range from 1 to 200 Hz corresponds to a change in the laser power within the range of approximately 10 mW to 2 W.

10. The hand piece as recited in one of the preceding claims,
wherein the control device (12) has a first converter for converting lengths of the second actuator travel into adequate pressure values of a medium as well as a second converter for converting the pressure values into adequate electrical control signals for a laser driving circuit (9).

11. The hand piece according to the definition of the species in claim 1, however,
additionally equipped with a suction cannula for removing tissue particles ablated by the action of a laser, the suction cannula being connected to a suction device whose suction performance is able to be preselected and changed,
characterized by a start/stop signal transmitter, which is integrated in the hand piece (1), for the suction device as well as at least one control device, which is/are also integrated in the hand piece (1), for preselecting and/or changing the suction performance.

12. The hand piece as recited in claim 11,
wherein the hand piece (1) has both a control device for preselecting and/or changing at least one physical parameter of the laser radiation and at least one control device for preselecting and/or changing the suction performance.

13. The hand piece as recited in one of the preceding claims,
wherein the hand piece (1) is designed for one-handed manipulation, in connection with which the functions of holding, aligning the laser beam with a preselected target site and actuating the existing control devices are executable using the fingers of one hand.

14. The hand piece as recited in one of the preceding claims,
wherein the start/stop signal transmitter(s) and the control devices (12) are designed as a unit which is frictionally and/or positively connected to the remaining parts of the hand piece (1) and detachable by hand force.

15. The hand piece as recited in claim 14,
wherein the unit which is detachable from the hand piece (1), is made from materials which can be subjected to a sterilization process, preferably to a sterilization using moist heat, without the risk of damage.
